(19) United States
(12) Patent Application Publication
(10) Pub. No.: US 2009/0270444 A1
(43) Pub. Date: Oct. 29, 2009

(54) 1,7-Naphthyridines

(76) Inventors: Colin David Eldred, Hertfordshire (GB); John Edward Robinson, Hertfordshire (GB); Alison Judith Steel, Hertfordshire (GB)

Correspondence Address: GLAXOSMITHKLINE;CORPORATE INTELLECTUAL PROPERTY, MAI B482, FIVE MOORE DR., PO BOX 13398, RESEARCH TRIANGLE PARK, NC, 27709-3398, US

(21) Appl. No.: 11/718,892

(22) PCT Filed: Nov. 17, 2005
(86) PCT No.: PCT/EP05/12462

§ 371 Date: Jan. 16, 2008

(30) Foreign Application Priority Data
Nov. 19, 2004 (GB) 0425572.5

(51) Int. Cl. A61K 31/4375; C07D 471/04; A61P 37/08

(52) U.S. Cl. 514/300; 546/122

(57) ABSTRACT

There are provided according to the invention novel compounds of formula (I) and pharmaceutically acceptable salts and solvates thereof,

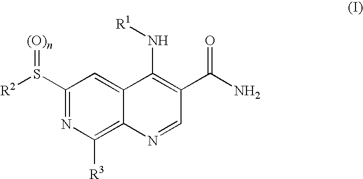

wherein:
R¹ is phenyl which may be unsubstituted or substituted by one or two substituents selected from fluorine, chlorine, C₁₋₂alkoxy-, —CN; phenyl fused to a 5-membered saturated ring containing one oxygen atom; pyridinyl which may be unsubstituted or substituted by one or two substituents selected from fluorine or chlorine; or C-linked pyrazolyl which may be unsubstituted or substituted by the substituent C₁₋₂alkyl;
R² is C₁₋₄alkyl;
R³ is C₁₋₂alkyl; and
n is 0, 1 or 2.

The present invention relates to 1,7-naphthyridine compounds, processes for their preparation, intermediates usable in these processes, and pharmaceutical compositions containing the compounds. The invention also relates to the use of the 1,7-naphthyridine compounds in therapy, for example as inhibitors of phosphodiesterases and/or for the treatment and/or prophylaxis of inflammatory and/or allergic diseases such as chronic obstructive pulmonary disease (COPD), asthma, rheumatoid arthritis or allergic rhinitis.

It is desirable to find new compounds which bind to, and preferably inhibit, phosphodiesterase type IV (PDE4).

According to the invention there is provided a compound of formula (I) or a pharmaceutically acceptable salt or solvate thereof:

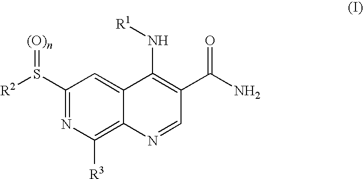

wherein:
R¹ is phenyl which may be unsubstituted or substituted by one or two substituents selected from fluorine, chlorine, C₁₋₂alkoxy-, —CN; phenyl fused to a 5-membered saturated ring containing one oxygen atom; pyridinyl which may be unsubstituted or substituted by one or two substituents selected from fluorine or chlorine; or C-linked pyrazolyl which may be unsubstituted or substituted by the substituent C₁₋₂alkyl;
R² is C₁₋₄alkyl;
R³ is C₁₋₂alkyl; and
n is 0, 1 or 2.

As used herein, the term “alkyl” refers to straight or branched hydrocarbon chains containing the specified number of carbon atoms. For example, C₁₋₄alkyl means a straight or branched alkyl chain containing at least 1, and at most 4, carbon atoms.

Examples of “alkyl” as used herein include, but are not limited to, methyl, ethyl, n-propyl, iso-propyl, n-butyl, sec-butyl, tert-butyl.

As used herein, the term “substituted” refers to substitution with the named substituent or substituents, multiple degrees of substitution being allowed unless otherwise stated.

In one embodiment R¹ is phenyl fused to a 5-membered saturated ring containing one oxygen atom.

In another embodiment R² is methyl.

In another embodiment R³ is methyl.

In another embodiment:

R¹ is phenyl fused to a 5-membered saturated ring containing one oxygen atom;
R² is methyl;
R³ is methyl; and
n is 0, 1 or 2.

In another embodiment:

R¹ is phenyl which may be unsubstituted or substituted by one or two substituents selected from fluorine, chlorine, C₁₋₂alkoxy-, —CN;
R² is methyl;
R³ is methyl; and
n is 0, 1 or 2.

In another embodiment:

R¹ is pyridinyl which may be unsubstituted or substituted by one or two substituents selected from fluorine or chlorine;
R² is methyl;
R³ is methyl; and
n is 0, 1 or 2.

In another embodiment:

R¹ is C-linked pyrazolyl which may be unsubstituted or substituted by the substituent C₁₋₂alkyl;
R² is methyl;
R³ is methyl; and
n is 0, 1 or 2.

It is to be understood that the present invention covers all combinations of substituent groups referred to herein above.

It is to be understood that the present invention covers all combinations of particular and preferred groups described herein above.

Particular compounds according to the invention include those mentioned in the examples and their pharmaceutically acceptable salts and solvates. Specific examples which may be mentioned include:

### EXAMPLE 1

### 4-(2,3-Dihydro-1-benzofuran-4-ylamino)-8-methyl-6-(methylthio)-1,7-naphthyridine-3-carboxamide

### EXAMPLE 2

### 4-(2,3-Dihydro-1-benzofuran-4-ylamino)-8-methyl-6-(methylsulfonyl)-1,7-naphthyridine-3-carboxamide

### EXAMPLE 3

### (±) 4-(2,3-Dihydro-1-benzofuran-4-ylamino)-8-methyl-6-(methylsulfinyl)-1,7-naphthyridine-3-carboxamide

and pharmaceutically acceptable salts and solvates thereof.

Salts of the compounds of the present invention are also encompassed within the scope of the invention. Because of their potential use in medicine, the salts of the compounds of formula (I) are preferably pharmaceutically acceptable. Suitable pharmaceutically acceptable salts can include acid addition salts. A pharmaceutically acceptable acid addition salt can be formed by reaction of a compound of formula (I) with a suitable inorganic or organic acid (such as hydrobromic, hydrochloric, sulfuric, nitric, phosphoric, p-toluenesulfonic, methanesulfonic or naphthalenesulfonic acid), optionally in a suitable solvent such as an organic solvent, to give the salt which is usually isolated for example by crystallisation and filtration. A pharmaceutically acceptable acid addition salt of a compound of formula (I) can be for example a hydrobromide, hydrochloride, sulfate, nitrate, phosphate, p-toluenesulfonate, methanesulfonate or naphthalenesulfonate salt. Other non-pharmaceutically acceptable salts, eg. trifluoroacetates, may be used, for example in the isolation of compounds of the invention, and are included within the scope of this invention. The invention includes within its scope all possible stoichiometric and non-stoichiometric forms of the salts of the compounds of formula (I).

Those skilled in the art of organic chemistry will appreciate that many organic compounds can form complexes with solvents in which they are reacted or from which they are precipitated or crystallized. These complexes are known as “solvates”. For example, a complex with water is known as a “hydrate”. Solvates of the compound of the invention are within the scope of the invention.

Certain compounds of formula (I) may exist in stereoisomeric forms (e.g. they may contain one or more asymmetric carbon or sulphur atoms or may exhibit cis-trans isomerism). The individual stereoisomers (enantiomers and diastereomers) and mixtures of these are included within the scope of the present invention. The present invention also covers the individual isomers of the compounds represented by formula (I) as mixtures with isomers thereof in which one or more chiral centres are inverted. Likewise, it is understood that compounds of formula (I) may exist in tautomeric forms other than that shown in the formula and these are also included within the scope of the present invention.

The compounds of this invention may be made by a variety of methods, including standard chemistry. Any previously defined variable will continue to have the previously defined meaning unless otherwise indicated. Illustrative general synthetic methods are set out below and then specific compounds of the invention are prepared in the working Examples.

### Process a

Compounds of formula (I), wherein R¹, R² and R³ are as defined above and n is 0, 1 or 2, may be prepared as outlined in scheme 1.

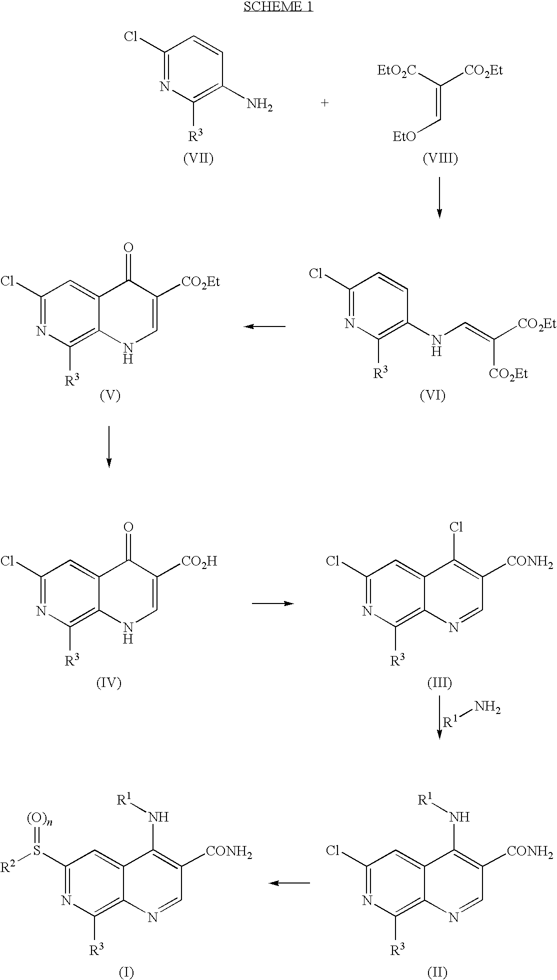

Referring to scheme 1, compounds of formula (I), wherein R¹, R² and R³ are as defined above and n is 0, may be prepared from compounds of formula (II);

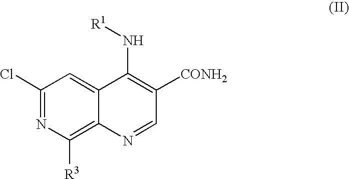

wherein R¹ and R³ are as defined above, by treatment with a suitable metal thioalkoxide, such as a sodium thioC₁₋₄alkoxide, in a suitable solvent for example N-methyl-2-pyrrolidinone, under microwave irradiation, at a suitable temperature, for example at 150° C., achieved using a suitable power, for example 20-200 W.

Compounds of formula (I), wherein R¹, R² and R³ are as defined above and n is 1 or 2, may be prepared from compounds of formula (I), wherein R¹, R² and R³ are as defined above and n is 0, by treatment with a suitable oxidising agent such as Oxone®, in a suitable solvent such as N,N-dimethylformamide, at a suitable temperature, for example room temperature.

Compounds of formula (II), wherein R¹ and R³ are as defined above, may be prepared from compounds of formula (III);

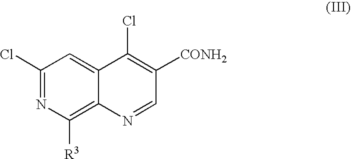

wherein R³ is as defined above, by treatment with an amine of formula R¹NH₂, wherein R¹ is as defined above, in a suitable solvent such as acetonitrile, at a suitable temperature, for example 85° C.

Compounds of formula (III), wherein R³ is as defined above, may be prepared from compounds of formula (IV);

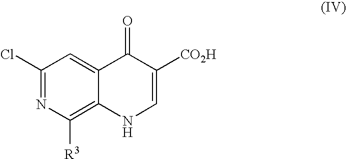

wherein R³ is as defined above, by treatment with a suitable chlorinating agent such as phosphorus oxychloride, at a suitable temperature, for example 50° C., followed by treatment with ammonia under suitable conditions such as 2M ammonia in methanol, optionally in the presence of a suitable solvent such as tetrahydrofuran, at a suitable temperature, for example between −78° C. and room temperature.

Compounds of formula (IV), wherein R³ is as defined above, may be prepared from compounds of formula (V);

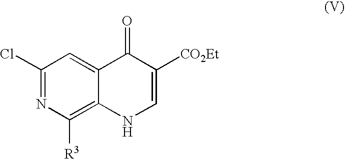

wherein R³ is as defined above, by treatment with a suitable base such as aqueous sodium hydroxide, in a suitable solvent such as ethanol, at a suitable temperature, for example 70° C.

Compounds of formula (V), wherein R³ is as defined above, may be prepared from compounds of formula (VI);

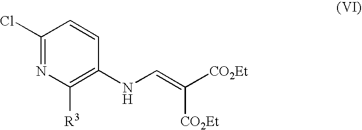

wherein R³ is as defined above, by heating in a suitable solvent such as diphenyl ether, at a suitable temperature, for example 240° C.

Compounds of formula (VI), wherein R³ is as defined above, may be prepared from compounds of formula (VII) wherein R³ is as defined above, and the compound of formula (VIII);

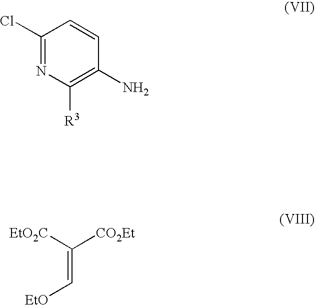

Suitable conditions include heating together the compounds of formulae (VII) and (VIII) in the absence of solvent, at a suitable temperature, for example 130° C.

### Process b

Compounds of formula (I) may also be prepared via interconversion of a compound of formula (I) into another compound of formula (I) using standard interconversion techniques such as those described in ‘Comprehensive Organic Transformations: a guide to functional group preparations’ by Richard Larock (VCH, 1989), incorporated herein by reference.

### Process c

Compounds of formula (I) may also be prepared by a process of deprotection of protected derivatives of compounds of formula (I). Examples of suitable protecting groups and the means for their removal can be found in T. W. Greene & P. G. M. Wuts ‘Protective Groups in Organic Synthesis’ (3^{rd} edition, J. Wiley and Sons, 1999).

The present invention also provides a compound of formula (I) or a pharmaceutically acceptable salt thereof for use as an active therapeutic substance in a mammal such as a human. The compound or salt may be useful in the treatment and/or prophylaxis of any of the conditions described herein and/or useful as a phosphodiesterase inhibitor, e.g. for use as a phosphodiesterase 4 (PDE4) inhibitor. “Therapy” may include treatment and/or prophylaxis.

Also provided is the use of a compound of formula (I) or a pharmaceutically acceptable salt thereof in the manufacture of a medicament (e.g. pharmaceutical composition) for the treatment and/or prophylaxis of an inflammatory and/or allergic disease in a mammal such as a human.

Also provided is a method of treatment and/or prophylaxis of an inflammatory and/or allergic disease in a mammal (e.g. human) in need thereof, which comprises administering to the mammal (e.g. human) a therapeutically effective amount of a compound of formula (I) as herein defined or a pharmaceutically acceptable salt thereof.

Phosphodiesterase 4 inhibitors are believed to be useful in the treatment and/or prophylaxis of a variety of diseases, especially inflammatory and/or allergic diseases, in mammals such as humans, for example: asthma, chronic bronchitis, emphysema, atopic dermatitis, urticaria, allergic rhinitis (seasonal or perennial), vasomotor rhinitis, nasal polyps, allergic conjunctivitis, vernal conjunctivitis, occupational conjunctivitis, infective conjunctivitis, eosinophilic syndromes, eosinophilic granuloma, psoriasis, rheumatoid arthritis, chronic obstructive pulmonary disease (COPD) including chronic bronchitis and emphysema, septic shock, ulcerative colitis, Crohn's disease, reperfusion injury of the myocardium and brain, chronic glomerulonephritis, endotoxic shock, adult respiratory distress syndrome, multiple sclerosis, or memory impairment (including Alzheimer's disease) pain or depression.

In the treatment and/or prophylaxis, the inflammatory and/or allergic disease is preferably chronic obstructive pulmonary disease (COPD) including chronic bronchitis and emphysema, asthma, rheumatoid arthritis, or allergic rhinitis, atopic dermatitis or psoriasis in a mammal (e.g. human). More preferably, the treatment and/or prophylaxis is of COPD including chronic bronchitis and emphysema, or asthma or allergic rhinitis in a mammal (e.g. human). PDE4 inhibitors are thought to be effective in the treatment of asthma (e.g. see M. A. Giembycz, Drugs, February 2000, 59(2), 193-212; Z. Huang et al., Current Opinion in Chemical Biology, 2001, 5, 432-438; and refs cited therein) and COPD (e.g. see S. L. Wolda, Emerging Drugs, 2000, 5(3), 309-319; Z. Huang et al., Current Opinion in Chemical Biology, 2001, 5, 432-438; and refs cited therein). COPD is often characterised by the presence of airflow obstruction due to chronic bronchitis and/or emphysema (S. L. Wolda, Emerging Drugs, 2000, 5(3), 309-319).

PDE4 inhibitors are thought to be effective in the treatment of allergic rhinitis (e.g. see B. M. Schmidt et al., J. Allergy & Clinical Immunology, 108(4), 2001, 530-536).

PDE4 inhibitors are thought to be effective in the treatment of rheumatoid arthritis and multiple sclerosis (e.g. see H. J. Dyke et al., Expert Opinion on Investigational Drugs, January 2002, 11(1), 1-13; C. Burnouf et al., Current Pharmaceutical Design, 2002, 8(14), 1255-1296; and A. M. Doherty, Current Opinion Chem. Biol., 1999, 3(4), 466-473; and refs cited therein). See e.g. A. M. Doherty, Current Opinion Chem. Biol., 1999, 3(4), 466-473 and refs cited therein for atopic dermatitis use.

PDE4 inhibitors have been suggested as having analgesic properties and thus being effective in the treatment of pain (A. Kumar et al., Indian J. Exp. Biol., 2000, 38(1), 26-30).

In the invention, the treatment and/or prophylaxis can be of cognitive impairment e.g. cognitive impairment in a neurological disorder such as Alzheimer's disease. For example, the treatment and/or prophylaxis can comprise cognitive enhancement e.g. in a neurological disorder. See for example: H. T. Zhang et al. in: Psychopharmacology, June 2000, 150(3), 311-316 and Neuropsychopharmacology, 2000, 23(2), 198-204; and T. Egawa et al., Japanese J. Pharmacol., 1997, 75(3), 275-81.

PDE4 inhibitors such as rolipram have been suggested as having antidepressant properties (e.g. J. Zhu et al., CNS Drug Reviews, 2001, 7(4), 387-398; O'Donnell, Expert Opinion on Investigational Drugs, 2000, 9(3), 621-625; and H. T. Zhang et al., Neuropsychopharmacology, October 2002, 27(4), 587-595).

For use in medicine, the compounds of the present invention are usually administered as a pharmaceutical composition.

The present invention therefore provides in a further aspect a pharmaceutical composition comprising a compound of formula (I) or a pharmaceutically acceptable salt thereof and one or more pharmaceutically acceptable carriers and/or excipients.

The pharmaceutical composition can be for use in the treatment and/or prophylaxis of any of the conditions described herein.

The compounds of formula (I) and/or the pharmaceutical composition may be administered, for example, by oral, parenteral (e.g. intravenous, subcutaneous, or intramuscular), inhaled, nasal, transdermal or rectal administration, or as topical treatments (e.g. lotions, solutions, creams, ointments or gels). Accordingly, the pharmaceutical composition is preferably suitable for oral, parenteral (e.g. intravenous, subcutaneous or intramuscular), topical, inhaled or nasal administration. More preferably, the pharmaceutical composition is suitable for topical, inhaled or oral administration, e.g. to a mammal such as a human. Inhaled administration involves topical administration to the lung, e.g. by aerosol or dry powder composition.

A pharmaceutical composition suitable for oral administration can be liquid or solid; for example it can be a solution, a syrup, a suspension or emulsion, a tablet, a capsule or a lozenge.

A liquid formulation will generally consist of a suspension or solution of the compound or pharmaceutically acceptable salt in a suitable pharmaceutically acceptable liquid carrier(s), for example an aqueous solvent such as water, aqueous ethanol or aqueous glycerine, or an oil, or a non-aqueous solvent, such as a surfactant, such as polyethylene glycol or an oil. The formulation may also contain a suspending agent, preservative, flavouring and/or colouring agent.

A pharmaceutical composition suitable for oral administration being a tablet can comprise one or more pharmaceutically acceptable carriers and/or excipients suitable for preparing tablet formulations. Examples of such carriers include lactose and cellulose. The tablet can also or instead contain one or more pharmaceutically acceptable excipients, for example binding agents, lubricants such as magnesium stearate, and/or tablet disintegrants.

A pharmaceutical composition suitable for oral administration being a capsule can be prepared using encapsulation procedures. For example, pellets containing the active ingredient can be prepared using a suitable pharmaceutically acceptable carrier and then filled into a hard gelatin capsule. Alternatively, a dispersion, or suspension or solution can be prepared using any suitable pharmaceutically acceptable carrier, for example an aqueous solution, aqueous gum or an oil and the dispersion, or suspension or solution then filled into a soft or hard gelatin capsule.

The compounds of formula (I) and/or the pharmaceutical composition may be administered by a controlled or sustained release formulation as described in WO 00/50011.

A parenteral composition can comprise a solution or suspension of the compound or pharmaceutically acceptable salt in a sterile aqueous carrier or parenterally acceptable oil. Alternatively, the solution can be lyophilised; the lyophilised parenteral pharmaceutical composition can be reconstituted with a suitable solvent just prior to administration.

Compositions for nasal or inhaled administration may conveniently be formulated as aerosols, solutions, drops, gels or dry powders.

For compositions suitable and/or adapted for inhaled administration, it is preferred that the compound or salt of formula (I) is in a particle-size-reduced form, and more preferably the size-reduced form is obtained or obtainable by micronisation. The preferable particle size of the size-reduced (e.g. micronised) compound or salt is defined by a D50 value of about 0.5 to about 10 microns (for example as measured using laser diffraction).

Aerosol formulations, e.g. for inhaled administration, can comprise a solution or fine suspension of the active substance in a pharmaceutically acceptable aqueous or non-aqueous solvent. Aerosol formulations can be presented in single or multidose quantities in sterile form in a sealed container, which can take the form of a cartridge or refill for use with an atomising device or inhaler. Alternatively the sealed container may be a unitary dispensing device such as a single dose nasal inhaler or an aerosol dispenser fitted with a metering valve (metered dose inhaler) which is intended for disposal once the contents of the container have been exhausted.

Where the dosage form comprises an aerosol dispenser, it preferably contains a suitable propellant under pressure such as compressed air, carbon dioxide or an organic propellant such as a hydrofluorocarbon (HFC). Suitable HFC propellants include 1,1,1,2,3,3,3-heptafluoropropane and 1,1,1,2-tetrafluoroethane. The aerosol dosage forms can also take the form of a pump-atomiser. The pressurised aerosol may contain a solution or a suspension of the active compound. This may require the incorporation of additional excipients e.g. co-solvents and/or surfactants to improve the dispersion characteristics and homogeneity of suspension formulations. Solution formulations may also require the addition of co-solvents such as ethanol. Other excipient modifiers may also be incorporated to improve, for example, the stability and/or taste and/or fine particle mass characteristics (amount and/or profile) of the formulation.

For pharmaceutical compositions suitable and/or adapted for inhaled administration, it is preferred that the pharmaceutical composition is a dry powder inhalable composition. Such a composition can comprise a powder base such as lactose, glucose, trehalose, mannitol or starch, the compound of formula (I) or salt thereof (preferably in particle-size-reduced form, e.g. in micronised form), and optionally a performance modifier such as L-leucine or another amino acid, cellobiose octaacetate and/or metals salts of stearic acid such as magnesium or calcium stearate. Preferably, the dry powder inhalable composition comprises a dry powder blend of lactose and the compound of formula (I) or salt thereof. The lactose is preferably lactose hydrate e.g. lactose monohydrate and/or is preferably inhalation-grade and/or fine-grade lactose. Preferably, the particle size of the lactose is defined by 90% or more (by weight or by volume) of the lactose particles being less than 1000 microns (micrometres) (e.g. 10-1000 microns e.g. 30-1000 microns) in diameter, and/or 50% or more of the lactose particles being less than 500 microns (e.g. 10-500 microns) in diameter. More preferably, the particle size of the lactose is defined by 90% or more of the lactose particles being less than 300 microns (e.g. 10-300 microns e.g. 50-300 microns) in diameter, and/or 50% or more of the lactose particles being less than 100 microns in diameter. Optionally, the particle size of the lactose is defined by 90% or more of the lactose particles being less than 100-200 microns in diameter, and/or 50% or more of the lactose particles being less than 40-70 microns in diameter. Most importantly, it is preferable that about 3 to about 30% (e.g. about 10%) (by weight or by volume) of the particles are less than 50 microns or less than 20 microns in diameter. For example, without limitation, a suitable inhalation-grade lactose is E9334 lactose (10% fines) (Borculo Domo Ingredients, Hanzeplein 25, 8017 JD Zwolle, Netherlands).

Optionally, in particular for dry powder inhalable compositions, a pharmaceutical composition for inhaled administration can be incorporated into a plurality of sealed dose containers (e.g. containing the dry powder composition) mounted longitudinally in a strip or ribbon inside a suitable inhalation device. The container is rupturable or peel-openable on demand and the dose of e.g. the dry powder composition can be administered by inhalation via the device such as the DISKUS™ device, marketed by GlaxoSmithKline. The DISKUS™ inhalation device is for example described in GB 2242134 A, and in such a device at least one container for the pharmaceutical composition in powder form (the container or containers preferably being a plurality of sealed dose containers mounted longitudinally in a strip or ribbon) is defined between two members peelably secured to one another; the device comprises: a means of defining an opening station for the said container or containers; a means for peeling the members apart at the opening station to open the container; and an outlet, communicating with the opened container, through which a user can inhale the pharmaceutical composition in powder form from the opened container.

For application topically to the skin, the compound of formula (I) or a pharmaceutically acceptable salt thereof could be formulated as a suitable ointment containing the active compound suspended or dissolved in, for example, a mixture with one or more of the following: mineral oil, liquid petrolatum, white petrolatum, propylene glycol, polyoxyethylene polyoxypropylene compound, emulsifying wax and water. Alternatively, it could be formulated as a suitable lotion or cream, suspended or dissolved in, for example, a mixture of one or more of the following: mineral oil, sorbitan monostearate, a polyethylene glycol, liquid paraffin, polysorbate 60, cetyl esters wax, cetearyl alcohol, 2-octyldodecanol, benzyl alcohol and water.

In the pharmaceutical composition, each dosage unit for oral or parenteral administration preferably contains from 0.01 to 3000 mg, more preferably 0.5 to 1000 mg, of a compound of the formula (I) or a pharmaceutically acceptable salt thereof, calculated as the free base. Each dosage unit for nasal or inhaled administration preferably contains from 0.001 to 50 mg, more preferably 0.005 to 5 mg, of a compound of the formula (I) or a pharmaceutically acceptable salt thereof, calculated as the free base.

The pharmaceutically acceptable compounds or salts of the invention can be administered in a daily dose (for an adult patient) of, for example, an oral or parenteral dose of 0.01 mg to 3000 mg per day or 0.5 to 1000 mg per day, or a nasal or inhaled dose of 0.001 to 50 mg per day or 0.005 to 5 mg per day, of the compound of the formula (I) or a pharmaceutically acceptable salt thereof, calculated as the free base.

The compounds, salts and/or pharmaceutical compositions according to the invention may also be used in combination with one or more other therapeutically active agents, for example, a β₂ adrenoreceptor agonist, an anti-histamine, an anti-allergic agent, an anti-inflammatory agent (including a steroid), an anticholinergic agent or an antiinfective agent (e.g. antibiotics or antivirals).

The invention thus provides, in a further aspect, a combination comprising a compound of formula (I) or a pharmaceutically acceptable salt thereof with one or more other therapeutically active agents, for example, a 2-adrenoreceptor agonist, an anti-histamine, an anti-allergic agent, an anti-inflammatory agent (including a steroid), an anticholinergic agent or an antiinfective agent (e.g. antibiotics or antivirals).

Examples of β₂-adrenoreceptor agonists include salmeterol (e.g. as racemate or a single enantiomer such as the R-enantiomer), salbutamol, formoterol, salmefamol, fenoterol or terbutaline and salts thereof, for example the xinafoate salt of salmeterol, the sulphate salt or free base of salbutamol or the fumarate salt of formoterol. Long-acting β₂-adrenoreceptor agonists are preferred, especially those having a therapeutic effect over a 24 hour period such as salmeterol or formoterol.

Examples of anti-histamines include methapyrilene, or loratadine, cetirizine, desloratadine or fexofenadine.

Examples of anti-inflammatory steroids include fluticasone propionate and budesonide.

Examples of anticholinergic compounds which may be used in combination with a compound of formula (I) or a pharmaceutically acceptable salt thereof are described in WO 03/011274 A2 and WO 02/069945 A2/US 2002/0193393 A1 and US 2002/052312 A1. For example, anticholinergic agents include muscarinic M3 antagonists, such as ipratropium bromide, oxitropium bromide or tiotropium bromide.

Other suitable combinations include, for example, combinations comprising a compound of formula (I) or a pharmaceutically acceptable salt thereof together with other anti-inflammatory agents (e.g. anti-inflammatory corticosteroids, NSAIDs, leukotriene antagonists (e.g. montelukast), iNOS inhibitors, tryptase and elastase inhibitors, beta-2 integrin antagonists, chemokine antagonists such as CCR3 antagonists, and adenosine 2a agonists, 5-lipoxygenase inhibitors and antiinfective agents such as an antibiotic or an antiviral). An iNOS inhibitor is preferably for oral administration. Suitable iNOS inhibitors (inducible nitric oxide synthase inhibitors) include those disclosed in WO 93/13055, WO 98/30537, WO 02/50021, WO 95/34534 and WO 99/62875. Suitable CCR3 inhibitors include those disclosed in WO 02/26722.

The combinations referred to above may conveniently be presented for use in the form of a pharmaceutical composition and thus a pharmaceutical composition comprising a combination as defined above together with one or more pharmaceutically acceptable carriers and/or excipients represent a further aspect of the invention.

The individual compounds of such combinations may be administered either sequentially or simultaneously in separate or combined pharmaceutical compositions.

### Biological Test Methods

### PDE3. PDE4B, PDE4D. PDE5 and PDE6 Primary Assay Methods

The activity of the compounds can be measured as described below. Preferred compounds of the invention are selective PDE4 inhibitors, i.e. they inhibit PDE4 (e.g. PDE4B and/or PDE4D) more strongly than they inhibit other PDE's such as PDE3 and/or PDE5.

### PDE Enzyme Sources and Literature References

Human recombinant PDE4B, in particular the 2B splice variant thereof (HSPDE4B2B), is disclosed in WO 94/20079 and also in M. M. McLaughlin et al., “A low Km, rolipram-sensitive, cAMP-specific phosphodiesterase from human brain: cloning and expression of cDNA, biochemical characterisation of recombinant protein, and tissue distribution of mRNA”, J. Biol. Chem., 1993, 268, 6470-6476. For example, in Example 1 of WO 94/20079, human recombinant PDE4B is described as being expressed in the PDE-deficient yeast Saccharomyces cerevisiae strain GL62, e.g. after induction by addition of 150 uM CuSO₄, and 100,000×g supernatant fractions of yeast cell lysates are described for use in the harvesting of PDE4B enzyme.

Human recombinant PDE4D (HSPDE4D3A) is disclosed in P. A. Baecker et al., “Isolation of a cDNA encoding a human rolipram-sensitive cyclic AMP phoshodiesterase (PDE IV_{D})”, Gene, 1994, 138, 253-256.

Human recombinant PDE5 is disclosed in K. Loughney et al., “Isolation and characterisation of cDNAs encoding PDE5A, a human cGMP-binding, cGMP-specific 3′,5′-cyclic nucleotide phosphodiesterase”, Gene, 1998, 216, 139-147.

PDE3 may be purified from bovine aorta as described by H. Coste and P. Grondin, “Characterisation of a novel potent and specific inhibitor of type V phosphodiesterase”, Biochem. Pharmacol., 1995, 50, 1577-1585.

PDE6 may be purified from bovine retina as described by: P. Catty and P. Deterre, “Activation and solubilization of the retinal cGMP-specific phosphodiesterase by limited proteolysis”, Eur. J. Biochem., 1991, 199, 263-269; A. Tar et al. “Purification of bovine retinal cGMP phosphodiesterase”, Methods in Enzymology, 1994, 238, 3-12; and/or D. Srivastava et al. “Effects of magnesium on cyclic GMP hydrolysis by the bovine retinal rod cyclic GMP phosphodiesterase”, Biochem. J., 1995, 308, 653-658.

### Inhibition of PDE Activity: Fluorescence Polarisation (FP) Assay

The ability of compounds to inhibit PDE catalytic activity was determined by IMAP Fluorescence Polarisation (FP) assay (Molecular Devices Ltd code: R8062) in 384-well format. Test compounds (small volume, e.g. 0.5 μl, of solution in DMSO) were preincubated at ambient temperature in black 384-well microtitre plates (supplier: NUNC, code 262260) with PDE enzyme in 10 mM Tris-HCl buffer pH 7.2, 10 mM MgCl₂, 0.1% (w/v) bovine serum albumin, 0.05% NaN₃ for 10-30 minutes. The enzyme level was set so that reaction was linear throughout the incubation.

For the PDE3, PDE4B and PDE4D assays Fluorescein adenosine 3′,5′-cyclic phosphate (Molecular Devices Ltd code: R7091) was added to give ˜40 nM final concentration. For the PDE5 and PDE6 assays Fluorescein guanosine 3′,5′-cyclic phosphate (Molecular Devices Ltd code: R7090) was added to give ˜40 nM final concentration. Plates were mixed on an orbital shaker for 10 seconds and incubated at ambient temperature for 40 minutes. IMAP binding reagent (Molecular Devices Ltd code: R7207) was added (60 μl of a 1 in 400 dilution in binding buffer of the kit stock solution) to terminate the assay. Plates were allowed to stand at ambient temperature for 1 hour. The FP ratio of parallel to perpendicular light was measured using an Analyst™ plate reader (from Molecular Devices Ltd). For inhibition curves, 11 concentrations (0.5 nM-30 μM) of each compound were assayed; more potent compounds were assayed over lower concentration ranges (assay concentrations were generally between 30 μM and 50 fM). Curves were analysed using ActivityBase and XLfit (ID Business Solutions Limited). Results were expressed as pIC₅₀ values.

Biological Data obtained for the Examples described above (PDE3, PDE4B, PDE4D, PDE5 and PDE6 inhibitory activity) are as follows. All data is thought to be accurate to within ±0.5 of the values stated below.

| | PDE3 | PDE4B | PDE4D | PDE5 | PDE6 |
|---|---|---|---|---|---|
| Example | mean | mean | mean | mean | mean |
| No. | pIC₅₀ | pIC₅₀ | pIC₅₀ | pIC₅₀ | pIC₅₀ |
| 1 | | 9.5 | 9.5 | <5 | <5 |
| 2 | <6.2 | 9.9 | 9.7 | <5 | <5 |
| 3 | <5.6 | 9.4 | 9.2 | <5 | <5 |

Emesis: Many known PDE4 inhibitors cause emesis and/or nausea to greater or lesser extents (e.g. see Z. Huang et al., Current Opinion in Chemical Biology, 2001, 5, 432-438, see especially pages 433-434 and refs cited therein). Therefore, it would be preferable but not essential that a PDE4 inhibitory compound of the invention causes only limited or manageable emetic side-effects. Emetic side-effects can for example be measured by the emetogenic potential of the compound when administered to ferrets; for example one can measure the time to onset, extent, frequency and/or duration of vomiting and/or writhing in ferrets after oral or parenteral administration of the compound. See for example A. Robichaud et al., “Emesis induced by inhibitors of PDE IV in the ferret” Neuropharmacology, 1999, 38, 289-297, erratum Neuropharmacology, 2001, 40, 465-465.

All publications, including but not limited to patents and patent applications, cited in this specification are herein incorporated by reference as if each individual publication were specifically and individually indicated to be incorporated by reference herein as though fully set forth.

### EXAMPLES

In this section, “intermediates” represent syntheses of intermediate compounds intended for use in the synthesis of the “Examples”.

Abbreviations used herein:
HPLC high performance liquid chromatography
LC/MS liquid chromatography/mass spectroscopy
SPE solid phase extraction column. Unless otherwise specified the solid phase will be silica gel. It is thought that compounds isolated by SPE are free bases.
SCX solid phase extraction (SPE) column with benzene sulfonic acid residues immobilised on the solid phase (eg. IST Isolute™ columns). When eluting with ammonia/methanol, it is thought that compounds isolated by SCX are free bases.

### General Experimental Details

### LC/MS (Liquid Chromatography/Mass Spectroscopy)

Waters ZQ mass spectrometer operating in positive ion electrospray mode, mass range 100-1000 amu.

UV wavelength: 215-330 nm
Column: 3.3 cm×4.6 mm ID, 3 μm ABZ+PLUS
Flow Rate: 3 ml/min

### Injection Volume: 5 μl

Solvent A: 95% acetonitrile+0.05% formic acid
Solvent B: 0.1% formic acid+10 mM ammonium acetate
Gradient: Mixtures of Solvent A and Solvent B are used according to the following gradient profiles (expressed as % Solvent A in the mixture): 0% A/0.7 min, 0-100% A/3.5 min, 100% A/1.1 min, 100-0% A/0.2 min

### Mass Directed Automated Preparative HPLC Column, Conditions and Eluent

The preparative column used was a Supelcosil ABZplus (10 cm×2.12 cm internal diameter; particle size 5 μm)

UV detection wavelength: 200-320 nm
Flow rate: 20 ml/min

### Injection Volume: 0.5 ml

Solvent A: 0.1% formic acid
Solvent B: 95% acetonitrile+0.05% formic acid
Gradient systems: mixtures of Solvent A and Solvent B are used according to a choice of 5 generic gradient profiles (expressed as % Solvent B in the mixture), ranging from a start of 0 to 50% Solvent B, with all finishing at 100% Solvent B to ensure total elution.

It is thought that compounds isolated by this method are free bases.

Evaporation of Product Fractions after Purification

Reference to column chromatography, SPE, SCX and preparative HPLC purification includes evaporation of the product containing fractions to dryness by an appropriate method.

### Aqueous Ammonia Solutions

‘880 Ammonia’ or ‘0.880 ammonia’ refers to concentrated aqueous ammonia (specific gravity 0.880).

### Intermediates and Examples

All reagents not detailed in the text below are commercially available from established suppliers such as Sigma-Aldrich.

### Intermediate 1

### Diethyl {(E)-2-[(6-chloro-2-methyl-3-pyridinyl)amino]ethenyl}propanedioate

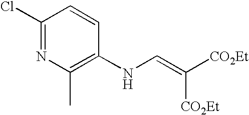

To 6-chloro-2-methyl-3-pyridinamine (8 g) (available from ACROS) was added diethyl [(ethyloxy)methylidene]propanedioate (11.9 ml) (available from Aldrich), and the mixture heated at 130° C. for 2 h. The cooled mixture was treated with cyclohexane (100 ml) and the solid filtered off to give the title compound as a cream solid (16.5 g).

LC/MS Rₜ 3.05 min m/z 313 [MH⁺].

### Intermediate 2

### Ethyl 6-chloro-8-methyl-4-oxo-1,4-dihydro-1,7-naphthyridine-3-carboxylate

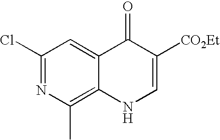

Intermediate 1 (5 g) was added portionwise to diphenyl ether (150 ml) at 240° C. over 5 min. The mixture was heated at 240° C. for 4 h, cooled and diluted with cyclohexane (250 ml). The resulting precipitate was filtered off to give the title compound as a dark brown solid (3.19 g).

LC/MS Rₜ 2.37 min m/z 267 [MH⁺].

### Intermediate 3

### 6-Chloro-8-methyl-4-oxo-1,4-dihydro-1,7-naphthyridine-3-carboxylic acid

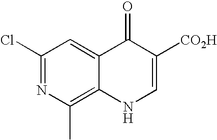

Intermediate 2 (3.19 g) in a mixture of 2M sodium hydroxide solution (30 ml) and ethanol (30 ml) was heated at 70° C. for 2 h. The ethanol was evaporated in vacuo, and the residue acidified to pH 2 with 2M hydrochloric acid. The resulting precipitate was isolated by filtration to give the title compound as a grey-brown solid (3.6 g).

LC/MS Rₜ 2.65 min m/z 239 [MH⁺].

### Intermediate 4

### 4,6-Dichloro-8-methyl-1,7-naphthyridine-3-carboxamide

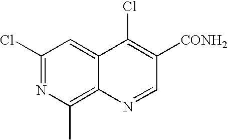

Intermediate 3 (2.1 g) was heated at 50° C. in phosphorus oxychloride (10 ml) overnight. The excess phosphorus oxychloride was evaporated in vacuo, and toluene (3×10 ml) was added and evaporated to give a semi-solid residue. The residue was dissolved in tetrahydrofuran (10 ml) and the solution added dropwise over 10 min to a solution of 2M ammonia in methanol (60 ml) at −78° C. After 1 h at −78° C. the mixture was allowed to warm to room temperature overnight and the precipitate filtered off and washed with water to give the title compound as a brown solid (0.94 g). Evaporation of the filtrate and purification of the residue by silica gel SPE, eluting with ethyl acetate, gave further title compound as a yellow solid (0.56 g).

LC/MS Rₜ 2.40 min m/z 256 [MH⁺].

### Intermediate 5

### 6-Chloro-4-(2,3-dihydro-1-benzofuran-4-ylamino)-8-methyl-1,7-naphthyridine-3-carboxamide

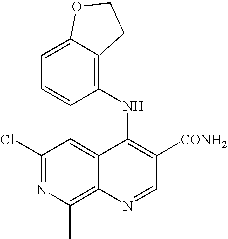

Intermediate 4 (5.35 g) was suspended in acetonitrile (50 ml), 2,3-dihydro-1-benzofuran-4-ylamine (J. Het. Chem (1980), 17(6), 1333-5) (3.1 g) was added, and the mixture was heated at 85° C. for 72 h. The mixture was filtered and the residue suspended in a mixture of water (100 ml) and saturated sodium carbonate solution (30 ml). After stirring for 1 h, the solid was filtered off and re-suspended in water (100 ml) and stirred for 15 minutes. The solid was filtered off and washed with water to give the title compound as a yellow solid (5.88 g).

LC/MS Rₜ 2.9 min m/z 355 [MH⁺].

### Example 1

### 4-(2,3-Dihydro-1-benzofuran-4-ylamino)-8-methyl-6-(methylthio)-1,7-naphthyridine-3-carboxamide

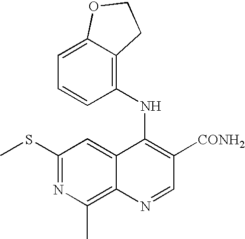

Intermediate 5 (0.06 g) was treated with sodium thiomethoxide (36 mg) in N-methyl-2-pyrrolidinone (2 ml), and the mixture heated under microwave irradiation at 150° C. for 2 h. The mixture was applied directly to an SCX ion exchange cartridge (2 g), and eluted with methanol followed by 10% 880 ammonia in methanol to give the crude product. Purification by mass directed preparative HPLC gave the title compound as a yellow solid.

LC/MS Rₜ 2.8 min m/z 367 [MH⁺].

### Example 2

### 4-(2,3-Dihydro-1-benzofuran-4-ylamino)-8-methyl-6-(methylsulfonyl)-1,7-naphthyridine-3-carboxamide

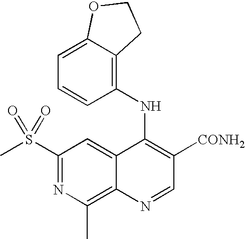

A solution of Example 1 (0.02 g) in N,N-dimethylformamide (1 ml) was treated with oxone (0.084 g), and the mixture was allowed to stand at room temperature overnight. The mixture was filtered and the filtrate evaporated under a stream of nitrogen. Purification by mass directed preparative HPLC gave the title compound as a yellow solid (0.004 g).

LC/MS Rₜ 2.5 min m/z 399 [MH⁺].

### Example 3

### (±)-4-(2,3-Dihydro-1-benzofuran-4-ylamino)-8-methyl-6-(methylsulfinyl)-1,7-naphthyridine-3-carboxamide

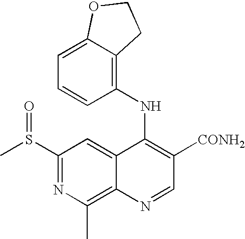

To a solution of Example 1 (0.02 g) in methanol (5 ml) at 0-5° C. was added a saturated solution of sodium periodate (0.012 g) in water dropwise over 5 min. After stirring for 4 h, oxone (0.030 g) was added, and the mixture was allowed to stand at room temperature overnight. The mixture was filtered and the filtrate evaporated in vacuo to give the crude product. Purification by mass directed preparative HPLC gave the title compound as a yellow solid (0.002 g).

LC/MS Rₜ 2.3 min m/z 383 [MH⁺].

What is claimed is:

1. A compound of formula (I) or a pharmaceutically acceptable salt or solvate thereof; 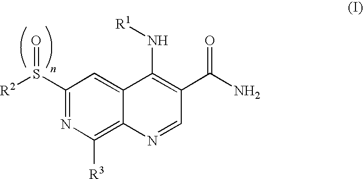
wherein:
R¹ is phenyl which may be unsubstituted or substituted by one or two substituents selected from fluorine, chlorine, C₁₋₂alkoxy-, —CN; phenyl fused to a 5-membered saturated ring containing one oxygen atom; pyridinyl which may be unsubstituted or substituted by one or two substituents selected from fluorine or chlorine; or C-linked pyrazolyl which may be unsubstituted or substituted by the substituent C₁₋₂alkyl;
R² is C₁₋₄alkyl;
R³ is C₁₋₂alkyl; and
n is 0, 1 or 2.

2. The compound according to claim 1 wherein:
R¹ is phenyl fused to a 5-membered saturated ring containing one oxygen atom;
R² is methyl;
R³ is methyl; and
n is 0, 1 or 2.

3. A compound selected from the group consisting of:
4-(2,3-Dihydro-1-benzofuran-4-ylamino)-8-methyl-6-(methylthio)-1,7-naphthyridine-3-carboxamide,
4-(2,3-Dihydro-1-benzofuran-4-ylamino)-8-methyl-6-(methylsulfonyl)-1,7-naphthyridine-3-carboxamide,
(±)4-(2,3-Dihydro-1-benzofuran-4-ylamino)-8-methyl-6-(methylsulfinyl)-1,7-naphthyridine-3-carboxamide
and pharmaceutically acceptable salts and solvates thereof.

4. A process for the preparation of a compound of formula (I) and pharmaceutically acceptable salts and solvates thereof as claimed in claim 1 which comprises;
(A) reacting a compound of formula (II) 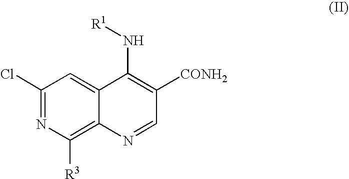
wherein R¹ and R³ are as defined above, with a suitable metal thioalkoxide, in a suitable solvent, under microwave irradiation, at a suitable temperature, achieved using a suitable power;
(B) interconversion of a compound of formula (I) into another compound of formula (I); or
(C) deprotecting a protected derivative of a compound of formula (I).

5. A method of treatment and/or prophylaxis of an inflammatory and/or allergic disease in a mammal in need thereof, which comprises administration of a therapeutically effective amount of a compound of formula (I) or a pharmaceutically acceptable salt or solvate thereof according to claim 1.

6. (canceled)

7. (canceled)

8. (canceled)

9. A pharmaceutical composition which comprises a compound according to claim 1 optionally with a pharmaceutically acceptable carrier or excipient.

10. A pharmaceutical composition according to claim 9 which is suitable for inhaled administration.

11. A pharmaceutical composition according to claim 9 which is suitable for oral administration.

12. The method of claim 5, wherein the mammal is human.

13. The method of claim 5, wherein the inflammatory and/or allergic disease is one for which a selective PDE4 inhibitor is indicated.

14. The process of claim 4, wherein the suitable metal thioalkoxide is sodium thioC₁₋₄alkoxide, the suitable solvent is N-methyl-2-pyrrolidinone, the suitable temperature is 150° C., and the suitable power is 20-200 W.
